(19) United States
(12) Patent Application Publication
(10) Pub. No.: US 2022/0072196 A1
(43) Pub. Date: Mar. 10, 2022

(54) MIXED MATERIAL IMPLANTS INCORPORATING ADDITIVES

(71) Applicant: CTL Medical Corporation, Addison, TX (US)

(72) Inventors: Sean SUH, Milltown, NJ (US); Jon SUH, Ambler, PA (US)

(21) Appl. No.: 17/471,878

(22) Filed: Sep. 10, 2021

Related U.S. Application Data
(60) Provisional application No. 63/076,852, filed on Sep. 10, 2020.

(51) Int. Cl. A61L 27/04; A61L 27/06; A61L 27/26; A61L 27/42; A61L 27/54

(52) U.S. Cl. CPC A61L 27/045; A61L 27/06; A61L 27/26; A61L 2430/38; A61L 27/54; A61L 2430/02; A61L 2430/12; A61L 27/425

(57) ABSTRACT

Disclosed are implants, devices and related manufacturing methods for implants comprising material mixtures including silicon nitride and/or other material additives in some of all of the implant body, including portions, layers and/or surface coatings thereof, for use as orthopedic implants such as joint and/or bone replacement implants used in in spinal surgeries, dental surgeries and/or other orthopedic procedures.

### CROSS-REFERENCE TO RELATED APPLICATIONS

This application claims priority to and benefit of U.S. Provisional Patent Application No. 63/076,852 entitled “Injection Molded Implants” filed Sep. 10, 2020, the disclosure of which is incorporated by reference herein in its entirety.

### FIELD OF THE INVENTION

The present subject matter relates generally to implants and related devices comprising silicon nitride and/or other material additives in some of all of the implant body, including portions, layers and/or surface coatings thereof, including orthopedic implants such as joint and/or bone replacement implants used in in spinal surgeries, dental surgeries and/or other orthopedic procedures. More specifically, implants can be manufactured from material mixtures which include ceramics and/or other biocompatible/bioresorbable additives such as silicon nitride or other materials in various forms as a component, including powders, granules, particulates, portions, slurries, layers and/or coatings of solids and/or particulates, that may be useful in joint and/or bone replacement implants used in spinal surgeries, dental surgeries and/or other surgical procedures. In various embodiments, the material mixture may optionally assume an initial flowable and/or moldable stage (which may include processing steps to render the materials flowable and/or moldable for injection or other molding), followed by a thickened, more solidified, cooled and/or cured stage for an implant, which implant may be utilized immediately after production and/or may be further processed for eventual implantation or other surgical use.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Various physical conditions can manifest themselves in the form of damage or degeneration of an intervertebral disc, the result of which is mild to severe chronic back pain. Intervertebral discs serve as “shock” absorbers for the spinal column, absorbing pressure delivered to the spinal column. Additionally, they maintain the proper anatomical separation between two adjacent vertebrae. This separation is necessary for allowing both the afferent and efferent nerves to exit and enter, respectively, the spinal column. Alternatively, or in addition, there are several types of spinal curvature disorders. Examples of such spinal curvature disorders include, but need not be limited to, lordosis, kyphosis and scoliosis.

One technique of treating spinal disorders, in particular the degenerative, traumatic and/or congenital issues, is via surgical arthrodesis of the spine. This can be accomplished by removing the intervertebral disk and replacing it with implant(s) and/or bone and/or immobilizing the spine to allow the eventual fusion or growth of the bone across the disk space to connect the adjoining vertebral bodies together. The stabilization of the vertebra to allow fusion is often assisted by the surgically implanted device(s) to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function.

Complications of joint fusions and/or other procedures of the spine can include those applicable to any surgery such as bone and/or soft-tissue infection, wound dehiscence, and failure of fixation. Other complications which may be more specific to fusion procedures can include malalignment, proximal or distal joint deterioration, and delayed union or nonunion, including potential complications resulting from medical comorbidities, patient noncompliance, and/or inappropriate fixation. Accordingly, there is need for further improvement in surgical implants, and the present subject matter is such improvement

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with various aspects of the present subject matter, implant devices and/or components thereof are described that incorporate various additives, including but not limited to silicon nitride (i.e., Si3N4 and/or chemical analogues thereof) or other material additives in their construction, either distributed throughout the entirety of the implant as well as within components, portions, layers and/or surfaces thereof. In various embodiments, the silicon nitride or other material additives can provide a variety of improvements to the implant, such as where the additives may be highly osteo-inductive and/or osteoconductive and/or will desirably facilitate and/or promote implant fixation to adjacent living bone surfaces and/or may reduce and/or inhibit periprosthetic infection and/or bacterial adhesion to the surfaces and/or interior portions of the implant.

In various applications, existing implant designs and/or performance may be enhanced by the addition of one or more additives such as silicon nitride or other material additives, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or materials in combination with various additives, including the use with various 3D printable materials. In such cases, the employment of such material mixtures in implant construction may enhance the strength and/or durability of a desired implant design, as well as allow for improved surgical outcomes and/or greatly reduced complication rates.

In various embodiments described herein, a variety of manufacturing steps and/or processes may be performed to create a desired implant and/or component(s) thereof, including the use of different manufacturing processes to create a single implant and/or the employment of multiple different manufacturing processes to create components that may be ultimately assembled into a single implant. Such processes could include any combinations of one or more of the following: (1) casting, (2) molding (including injection molding), (3) subtractive machining (i.e., milling and drilling), (4) additive machining (i.e., additive 3D printing), (5) surface coating and sputtering, (6) surface embedding, and/or any combinations thereof.

If desired, implants could be constructed from a variety of modular components, including modular components comprising different materials. If desired, such modular components could be provided in a kit form for selection and/or assembly in a surgical theatre and/or in situ during a surgical procedure. If desired, various components may be removable and replaceable.

In accordance with various aspects of the present subject matter, materials that are capable of being injection molded and/or processed using other manufacturing methods are combined with a variety of additives, including (but not limited to) silicon nitride (i.e., Si₃N₄ and/or chemical analogues thereof) in their mixtures and/or composition, which may include the incorporation of silicon nitride powders, granules, particulates, portions, pebbles, blocks, layers and/or coatings of solids and/or particulates within the material mixture. In various embodiments, the mixture may comprise a liquid, powder, granular substance, paste, gel or dough, and in various embodiments may preferably harden and/or cool to a substantially solid solidified material after processing. The employment of injection molding or other well established manufacturing techniques in producing such implants and/or implant components will desirably dramatically reduce production cost (i.e., volumetric cost reductions) and/or significantly reduce production cycle times.

In at least one exemplary embodiment, an implant material such as poly (ether ether ketone) or “PEEK” material can be combined with various percentages by weight and/or volume of a ceramic material such as a silicon nitride material, which when mixed and processed can result in block, implant and/or other structure capable of implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

In at least one alternative embodiment, a mixed material can include various percentages by weight and/or volume of a powdered, granulated and/or fluidized silicon nitride material, which when mixed can create a flowable and/or moldable material which can be formed into a variety of shape and/or which can be injected into a mold, void or opening to partially and/or fully fill the mold, void or opening, wherein the material can harden into a shape which can be defined by the cavity in which it sits. This could include the injection into various anatomical locations as well as injection and/or introduction into implants and/or other devices prior to, during and/or after their implantation into a targeted patient anatomy (i.e., such as within the graft chamber of an intervertebral fusion implant), as well as manufacture of the implant and/or components thereof by injection molding.

In accordance with various aspects of the present subject matter, implants can incorporate silicon nitride (i.e., Si3N4 and/or chemical analogues thereof) and/or other supplemental materials in their construction, either in the entirety of the implant as well as components, portions, layers and/or surfaces thereof. In various embodiments, the silicon nitride material(s) and/or other materials will be highly osteo-inductive and/or osteoconductive and will desirably facilitate and/or promote fixation to adjacent living bone surfaces, while concurrently reducing and/or inhibiting periprosthetic infection and/or bacterial adhesion.

In various applications, the utility of silicon nitride as an implant material can be enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

If desired, implants can be constructed from a variety of modular components, including modular components comprising different materials and/or injectable or formable silicon nitride. If desired, such modular components could be provided in a kit form for selection and/or assembly in a surgical theatre and/or in situ during a surgical procedure. If desired, various components may be removable and replaceable.

Various surgical methods for preparing anatomical surfaces and/or for implanting or placement of the various devices and/or components described herein are also described, including the insertion and placement of implants between adjacent vertebrae of the spine as well as within bones and/or between bones and/or joint surfaces or other body locations.

In accordance with another aspect of the present subject matter, various methods for manufacturing devices and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings. It is to be appreciated that two copies of the drawings are provided; one copy with notations therein for reference to the text and a second, clean copy that possibly provides better clarity.

FIG. 1 illustrates one exemplary embodiment of a production process for a mixed material implant;

FIG. 2 depicts one exemplary embodiment of a mold utilized in a mixed material implant production process;

FIGS. 3A and 3B depict perspective views of implant structures incorporating resorbable silicon nitride granules;

FIGS. 4A and 4B depict the implant structures of FIGS. 3A and 3B after absorption; of some silicon nitride;

FIG. 5 depicts another embodiment of a production process for a mixed material implant;

FIG. 6A depicts a perspective view of a silicon nitride agglomeration in a material mixture formulation;

FIG. 6B depicts various exemplary geometries for resorbable silicon nitride granules mixed into a base material to enhance macro porosity;

FIGS. 7A through 7C depict various exemplary silicon nitride granular shapes;

FIG. 8 depicts an exemplary grain size distribution for silicon nitride granules for use in material mixture formulations;

FIG. 9 depicts various cross-sectional views of one embodiment of a mixed material implant with various exemplary silicon nitride insert geometries formed therein;

FIGS. 10 and 11 depict various implants made of PEEK, Titanium and material mixtures including silicon nitride;

FIG. 12 depicts another embodiment of a production process for a mixed material implant; and

FIG. 13 depicts another embodiment of a production process for a mixed material implant.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals may represent similar parts throughout the several views of the drawings. In addition, the following is a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In various embodiments, the terms “including,” “comprising” and variations thereof, as used in this disclosure, should be interpreted as “including, but not limited to,” unless expressly specified otherwise. The terms “a,” “an,” and “the,” as used in this disclosure, mean “one or more,” unless expressly specified otherwise.

In some embodiments, devices and/or device components that may be disclosed in communication with each other need not necessarily be in continuous communication with each other, unless expressly specified otherwise. In addition, components that are in direct contact with each other may contact each other directly or indirectly through one or more intermediary articles or devices. The device(s) disclosed herein may be made of a material such as silicon nitride, which may alternatively be combined, in various embodiments, with other materials such as, for example, a polymer, a metal, an alloy, or the like. For instance, a disclosed device(s) may comprise a silicon nitride additive material, alone or in combination with a Polyether Ether Ketone (PEEK), a titanium, a titanium alloy, or the like, or various combinations of the foregoing. The material may be formed by a process such as, for example, an active reductive process of a metal (e.g., titanium or titanium alloy) to increase the amount of nanoscaled texture to device surface(s), so as to increase promotion of bone growth and fusion.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single component, device and/or article is described herein, it will be readily apparent that more than one component, device and/or article may be used in place of a single component, device and/or article. Similarly, where more than one component, device and/or article is described herein, it will be readily apparent that a single component, device and/or article may be used in place of the more than one component, device and/or article. The functionality or the features of a component, device and/or article may be alternatively embodied by one or more other components, devices and/or articles which are not explicitly described as having such functionality or features.

Certain exemplary embodiments will now be described to provide an overall understanding of the principles of the structure, function, manufacture, and use of the components, devices and methods disclosed herein. One or more examples of these embodiments are illustrated in the accompanying drawings. Those of ordinary skill in the art will understand that the components, devices and/or methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various components, devices, systems and methods for treating various anatomical structures of the spine and/or other areas of human and/or animal bodies. While the disclosed embodiments may be particularly well suited for use during surgical procedures for the repair, fixation and/or support of vertebrae, it should be understood that various other anatomical locations of the body may benefit from various features of the present invention, including for the repair of bones and for use in, for example, orthopedic surgery, including vertebrae repair, musculoskeletal reconstruction, fracture repair, hip and knee reconstruction, osseous augmentation procedures and oral/maxillofacial surgery.

Various embodiments herein encompass implants that incorporate silicon nitride (i.e., Si₃N₄ and/or chemical analogues thereof) or other material additives in their mixtures and/or composition, which may include the incorporation of silicon nitride slurries, powders, granules, particulates, portions, pebbles, blocks, layers and/or coatings of solids and/or particulates within the material mixture.

In at least one exemplary embodiment, an implant could comprise a standard or commonly-accepted implant material which can be combined with various percentages by weight and/or volume of a ceramic material such as a silicon nitride material, which when mixed can be formed into a polymerized, formed, “cured” and/or cooled block, implant and/or structure capable of implantation in a bony defect and/or other location. In various embodiments, the ceramic material may comprise a granular or regularly/irregularly shaped material, with the granules having a plurality of interconnecting micropores. In various embodiments, a plurality of different sizes of granules may be used.

In various embodiments, implants can be formed that incorporate silicon nitride and/or resorbable granular materials such as calcium phosphate or other materials, which may facilitate bone ingrowth, bone outgrowth and/or bone through-growth in varying amounts. Such implants would desirably provide the improved bacteriostatic properties of silicon nitride, and also allow for superior adhesions and/or anchoring to surrounding structures. Concurrently, the underlying base or “scaffolding” material could desirably provide significant strength and/or durability for the implant. Moreover, unlike antibiotic-loaded implants currently available, the bacteriostatic properties of silicon nitride are not anticipated to appreciably fade or diminish over time, and the present of silicon nitride within the mixture does not markedly weaken the strength and/or durability of the underlying base material(s). In various embodiments the disclosed implants will desirably incorporate materials such as silicon nitride that are “phase stable” to a desired degree. For example, various embodiments will desirably withstand standard autoclave sterilization conditions such as 120° C. of 1 atmosphere steam for up to 100 hours of time, with no appreciable change in phase composition, no appreciable change in flexural strength and an inherently stable microstructure. Moreover, such materials will desirably provide favorable imaging characteristics, such as high levels of radiolucency and/or no significant MRI or CT scan artifacts.

In various embodiments, the properties of the disclosed implants will desirably include improvements in one or more of the following: (1) Flexibility in manufacturing and structural diversity, (2) Strong, tough and reliable constructs, (3) Phase stable materials, (4) Favorable imaging characteristics, (5) Hydrophilic surfaces and/or structures, (6) Osteoconductive, (7) Osteoinductive, (8) Anti-Bacterial characteristics, and/or (9) cost reductions and/or increases in manufacturing throughput.

In accordance with various aspects of the present subject matter, implants, devices and/or components thereof are described that incorporate silicon nitride (i.e., Si3N4 and/or chemical analogues thereof) in their construction, either in the entirety of the implant as well as components, portions, layers and/or surfaces thereof. In various embodiments, the silicon nitride material(s) will be highly osteo-inductive and/or osteoconductive and will desirably facilitate and/or promote fixation to adjacent living bone surfaces, while concurrently reducing and/or inhibiting periprosthetic infection and/or bacterial adhesion.

In various embodiments, the disclosed mixed materials and/or implants formed thereof will desirably provide one or more of the following advantages: (1) macro-interdigitation, (2) micro-interdigitation, (3) osteo-induction, (4) osteo-conduction, (5) hydrophilicity (i.e., due to macro, micro and/or chemical effects), (6) germicidal effects, (7) radiolucency or semi-radiolucency, (8) MRI safe characteristics and/or (9) CAT scan refractivity free characteristics.

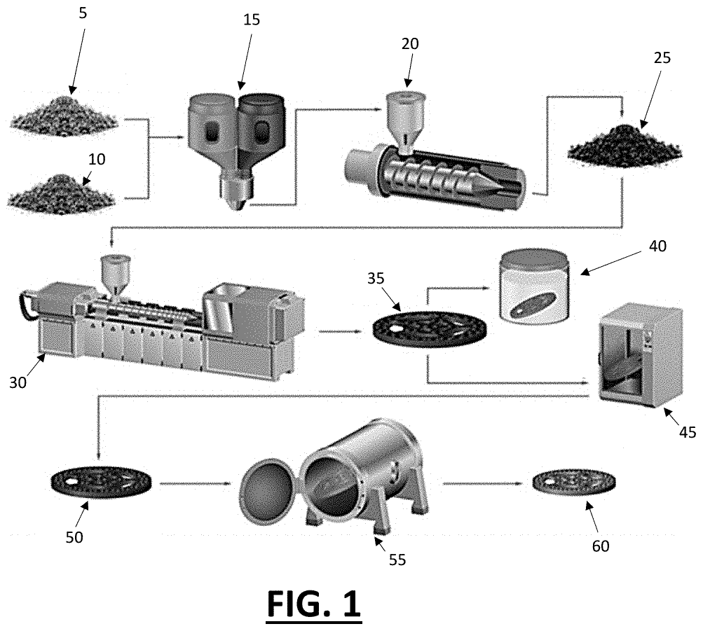

FIG. 1 depicts one exemplary embodiment of a production process for a mixed material implant, wherein a metal powder 5 and a silicon nitride powder 10 are mixed in a mixer 15, and then optionally processed and/or granulated in an extruder 20 or similar processing device. The granulator produces a feedstock 25 which can be introduced into an injection molding apparatus 30, which molding process can then produce a raw product 35, which can optionally be cleaned 40 and/or thermally debound 45. Where the raw product forms a green body 50, the green body may be sintered 55 or otherwise processed, with a final product 60 obtained therefrom.

In various embodiment, the mixed material implant may be post-processed after initial forming, such as during the “green state” off the implant. For example, prior to the sintering step, the mixed material implant maybe modified by removal and/or addition of material, including the addition of material containing additives by 3D additive manufacturing, and the modified implant can then be sintered as previously described. Where the implant may shrink or otherwise deform during the sintering process, the removal and/or addition of such material post-processing may accommodate such shape changes during the sintering process, resulting in a final implant with desired shape and/or size characteristics. Of course, such post-processing steps may optionally be undertaken at any point in the production process, including after finished production of the implant.

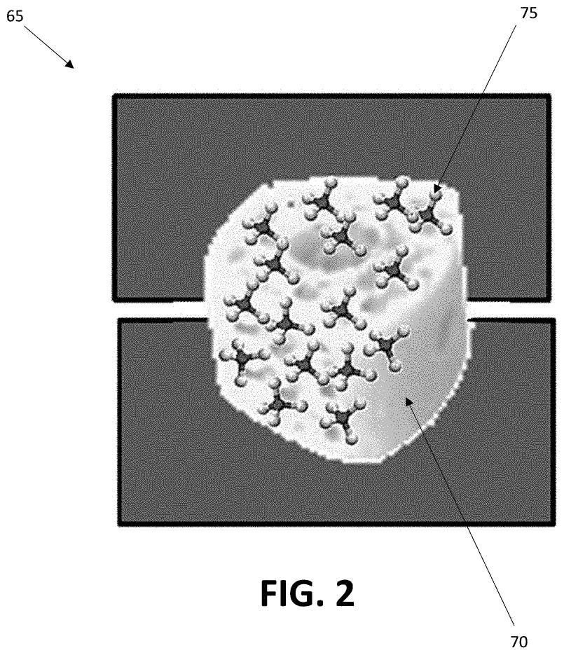

FIG. 2 depicts one exemplary embodiment of a mold 65 which can be utilized in an exemplary process to produce a mixed material; implant or other component, wherein the implant 70 can incorporate various additives 75, including silicon nitride and/or other materials described herein.

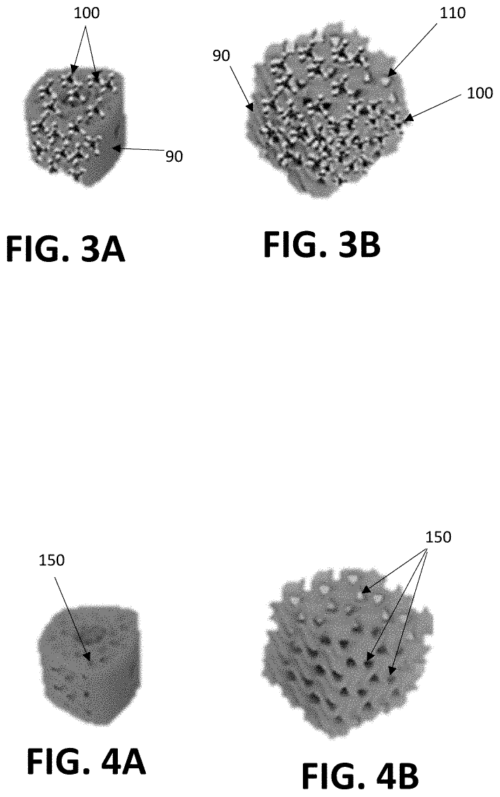

As best seen in FIGS. 3A and 3B, an implant can desirably comprise a PEEK base 90 with a plurality of silicon nitride granules 100 located therein. In various embodiments, the granules can be mixed with and suspended within a PEEK powder or other precursor, with the mixed material then being cast, injection molded and/or otherwise formed with various of the silicon nitride granules within the solid material being exposed to the surrounding anatomy. In various embodiments, such as shown in FIG. 3B, the PEEK base 90 can further optionally include openings and/or voids 110 formed therein. FIGS. 4A and 4B depict the mixed material implants of FIGS. 3A and 3B after partial resorption of silicon nitride granules near the surface of the blocks, wherein additional macro pores 150 have been formed as some of the silicon nitride granules have been resorbed and/or remodeled by the patient.

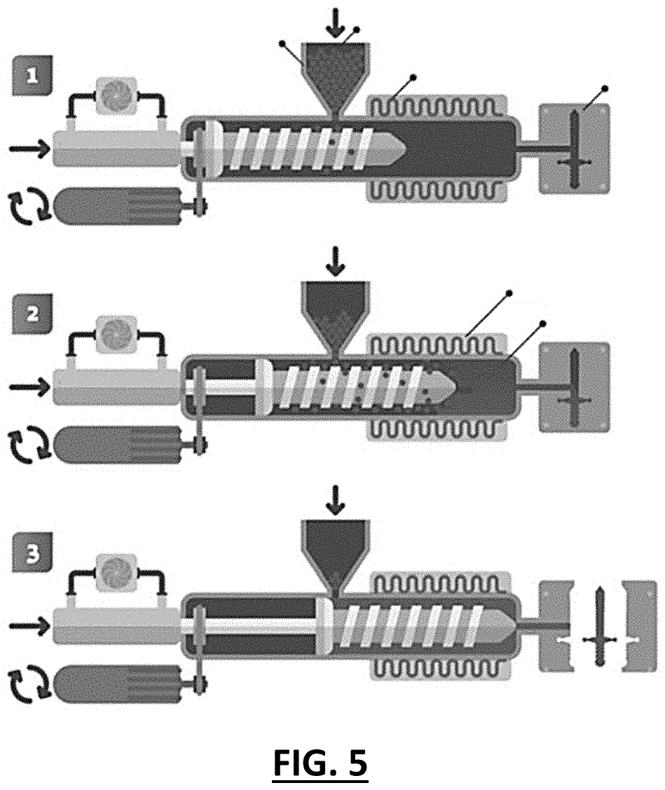

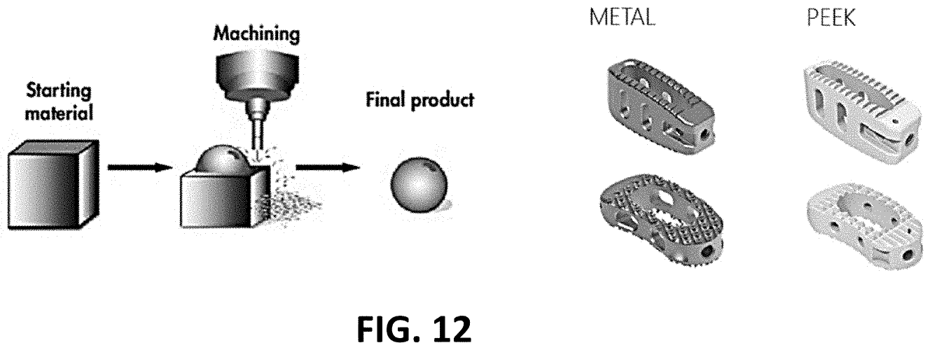

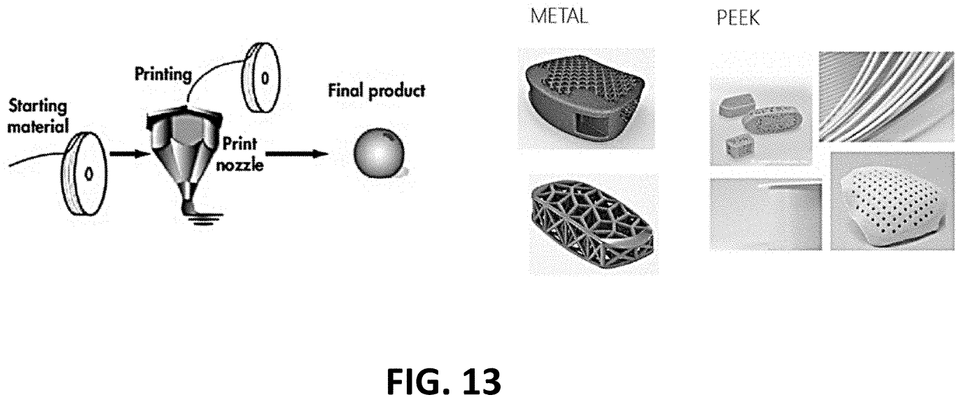

FIG. 5 depicts another production process for a mixed material implant, in which the mixed material granules are placed in a hopper of a screw-type extruder, wherein the material mixture is pressurized and/or heated in a known manner and then introduced in an implant mold, and the implant material cools within the mold and the mold can be removed to reveal a completed implant. FIGS. 12 and 13 depict additional embodiments of production processes that may be utilized to manufacture a mixed material implant (which may optionally be utilized alone or in combination with the various production processes described herein), including subtractive machining processes such as milling and drilling (FIG. 12) and additive machining processes such as 3d printing and layer manufacturing (FIG. 13).

In various embodiments, silicon nitride may be particularly well suited for inclusion into a material mixture with other base implant materials prior to manufacturing processes that utilize heat and/or pressure to form an implant. Silicon Nitride has a melting point of approximately 3,552 degrees Fahrenheit, while titanium melts at approximately 3,034 degrees F. and PEK melts at approximately 649.4 degrees F. An exemplary mixture of these materials (i.e., silicon nitride granules with titanium powder and/or silicon nitride powder with PEEK granules) can be heat treated and/or pressurized in various manufacturing processes to a point where the base implant material may anneal, melt and/or flow together while the silicon nitride additive remains solid or not melted, thereby facilitating the creation of a base implant with suspended silicon nitride particles dispersed therein.

In various applications, the utility of silicon nitride as a component of medical implants may be further enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

Silicon nitride (Si3N4) and its various analogs can impart both antibacterial and osteogenic properties to an implant, including to mixtures containing Si₃N₄ and/or bulk Si₃N₄ as well as to implants coated with layers of Si₃N₄ of varying thicknesses. In bone replacement as well as prosthetic joint fusion and/or replacement, osseous fixation of implants through direct bone ingrowth (i.e., cementless fixation) is often preferred, and such is often attempted using various surface treatments and/or the incorporation of porous surface layers (i.e., porous Ti₆Al₄V alloy) on one or more bone-facing surfaces of an implant. Silicon nitride surfaces and/or interior portions express reactive nitrogen species (RNS) that promote cell differentiation and osteogenesis, while resisting both gram-positive and gram-negative bacteria. This dual advantage of RNS in terms of promoting osteogenesis, while discouraging bacterial proliferation, can be of significant utility in a variety of implant designs.

Desirably, the inclusion of silicon nitride components into a given material mixture will encompass the use of granularized and/or powdered silicon nitride, as well as bulk silicon nitride, as well as implants incorporating other materials that may also include silicon nitride components and/or layers therein, with the silicon nitride becoming an active agent of bone fusion. RNS such as N₂O, NO, and —OONO are highly effective biocidal agents, and the unique surface chemistries of Si₃N₄ facilitate its activity as an exogenous NO donor. Spontaneous RNS elution from Si₃N₄ discourages surface bacterial adhesion and activity, and unlike other direct eluting sources of exogenous NO, Si₃N₄ elutes mainly NH₄₊ and a small fraction of NH₃ ions at physiological pH, because of surface hydrolysis and homolytic cleavage of the Si—N covalent bond. Ammonium NH₄₊ can enter the cytoplasmic space of cells in controlled concentrations and through specific transporters, and is a nutrient used by cells to synthesize building-block proteins for enzymes and genetic compounds, thus sustaining cell differentiation and proliferation. Together with the leaching of orthosilicic acid and related compounds, NH₄₊ promotes osteoblast synthesis of bone tissue and stimulates collagen type 1 synthesis in human osteoblasts. Conversely, highly volatile ammonia NH₃ can freely penetrate the external membrane and directly target the stability of DNA/RNA structures in bacterial cells. However, the release of unpaired electrons from the mitochondria in eukaryotic cells activates a cascade of consecutive reactions, which starts with NH₃ oxidation into hydroxylamine NH₂OH (ammonia monooxygenase) along with an additional reductant contribution leading to further oxidation into NO₂₋ nitrite through a process of hydroxylamine oxidoreductase. This latter process involves nitric oxide NO formation. In Si₃N₄, the elution kinetics of such nitrogen species is slow but continuous, thus providing long-term efficacy against bacterial colonies including mutants (which, unlike eukaryotic cells, lack mitochondria). However, when slowly delivered, NO radicals have been shown to act in an efficient signaling pathway leading to enhanced differentiation and osteogenic activity of human osteoblasts. Desirably, Si₃N₄ materials can confer resistance against adhesion of both Gram-positive and Gram-negative bacteria, while stimulating osteoblasts to deposit more bone tissue, and of higher quality.

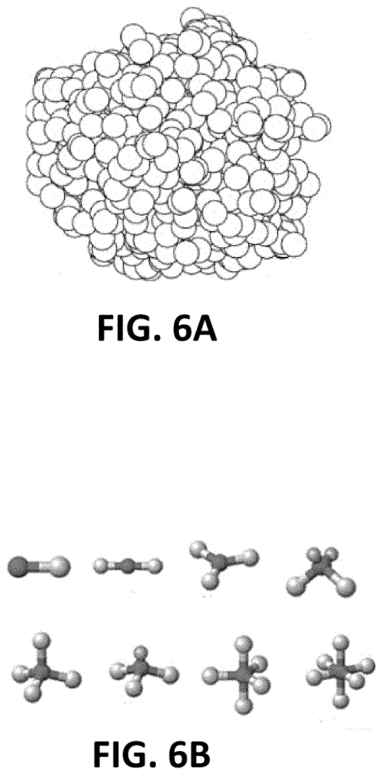

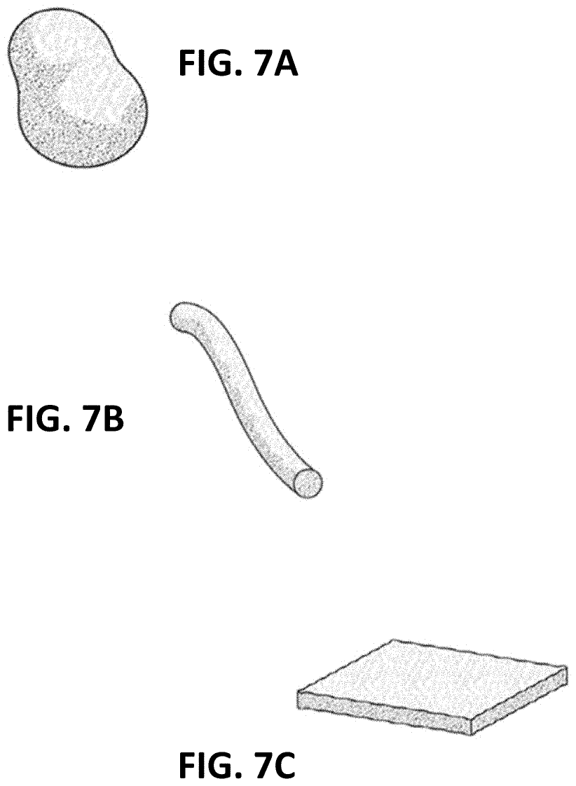

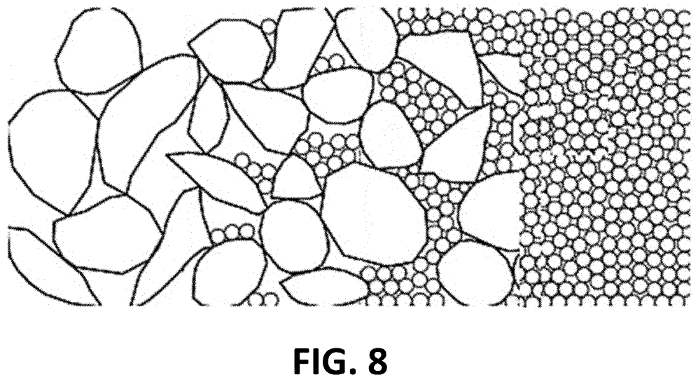

In another exemplary embodiment, disclosed are implant material mixtures which have both improved structural properties and improved osteoconductivity to regenerate and heal the host bone tissue. In this embodiment, the distribution of a granulated microporous ceramic material such as silicon nitride will desirably provide improved structural properties, whilst the microporous structure of the ceramic material granules allows host tissue to bind and regenerate around and within the material mixture. In some embodiments the ceramic material granules may comprise a single average size granule or granule distribution (See FIG. 6A), while in other embodiments, the granule size may be widely distributed and/or essentially random within a range of sizes (See FIG. 8). In another alternative embodiment, at least two different preselected sizes, or ranges of sizes, of granulated material can be used, e.g. in a similar manner to sand and gravel being used to make concrete. The different size of the silicon nitride “sand and gravel” may be helpful in improving the strength of the material. Preferably, the ceramic material will be generally evenly distributed throughout a cross-section of the bulk material, that is substantially without clumps of ceramic material forming. If desired, the various silicon nitride granules may comprise a variety of different shapes, including rounded particles, irregularly shaped particles (see FIG. 7A), elongated particles, fibers or “strings” (see FIG. 7B), flattened or planar particles (see FIG. 7C), or other shapes, or any combination thereof. In many cases, multiple shapes and/or sizes of particles may provide for optimized packing and/or density of the silicon nitride material for certain applications.

If desired, a variety of sizes and/or shapes of silicon nitride granules and/or particles may be utilized in various embodiments of the present invention, which can include particles and/or microparticles that form a variety of geometric bonds and/or matrix shapes, including linear, trigonal planar, bent or angular, tetrahedral, trigonal pyramidal, trigonal bipyramidal, octahedral, and/or other shapes, including those depicted in FIG. 6B.

In various embodiments, the individual granules of the silicon nitride material may have micropores. Preferably, the micropores are interconnecting. They are preferably not confined to the surface of the granules but are found substantially throughout the cross-section of the granules. Preferably, the diameter of the granule particles is between 10 μm and 1 mm, preferably 400 μm and 1000 μm, especially 500-900 μm, 500-800 μm or 600-700 μm. The ceramic material granules may be formed from a fused block of biomaterial by milling or grinding using, for example, a ball mill, and the size of the granules may be adjusted using, e.g. one or more sieves. In this manner, two or more different sized particles, or ranges of sizes of particles, may be obtained from a single “run” of the ball mill, if desired.

Where the silicon nitride granule size within a given material mixture is distributed between two different preselected sizes, or ranges of sizes, some embodiments may comprise a mixture of small and large granules. For example, the small granules may have a size range of 10 μm to 500 μm, especially 50 to 350 μm, most preferably 100 to 250 μm diameter, while the large granules may have a diameter of 250 μm to 1.5 mm, especially 500 μm to 1 mm, most preferably 600 μm to 800 μm.

In various embodiments, the implant material mixture may comprise a mixture of a carrier material such as titanium or PEEK with the ceramic material granules. That is, in the solidified bone substitute, the carrier forms a matrix that binds together the ceramic material granules.

A wide variety of material options may be selected for use in constructing implants and related components using material mixtures with silicon nitride and/or other materials, including (1) an implantable polymer and/or plastic such as PEEK, (2) implantable metals such as titanium, stainless steel, cobalt-chrome and various alloys thereof, (3) implantable ceramic materials, and/or (4) additives and/or composite mixtures and/or composite and/or compound forming surface treatments.

In various embodiments, an exemplary implant can comprise a ceramic silicon nitride in combination with a base material. If desired, the ceramic can comprise granules of a silicon nitride material, which is adhered and/or held within a bulk material matrix. Where the material mixture may be injected through a smaller diameter opening (i.e., during an injection molding process), the use of bulk and/or large granules of silicon nitride (or other material) implants may not be desirous and/or may be impractical. In such embodiments, it may be desirable to incorporate silicon nitride in a powdered, slurry and/or small granular form, to allow easy mixing with the bulk material. Desirably, the size of the granules allows it to be injected through the bore of injection molding component and/or screw-type extruder. For example, the preferable maximum size of silicon nitride granules and/or particles in such an application may be 0.1 mm, or 0.5 mm, or 1.0 mm, or 1.5 mm.

In various embodiments, silicon nitride granules in a material mixture can each include a plurality of micropores formed therein, with the micropores of varying shapes and/or sized within an individual granule. In various aspects of the invention, the ceramic granules will each include a plurality of micropores formed therein. In some aspects, the invention provides: a bone substitute comprising a mixture of a carrier material and silicon nitride material granules, the silicon nitride granules having a plurality of micropores of an average diameter of between 1 μm and 10 μm and/or between 10 μm and 50 μm and/or between 50 μm and 100 μm. Of course, smaller and/or larger pore sizes within the granules may have particular utility in certain applications.

In various embodiments, the volume and/or weight ratio of silicon nitride to other implant materials may be 1000:1, 100:1, 50:1, 10:1, 5:1, 2:1, 1:1, 1:2, 1:5, 1:10, 1:50. 1:100 and/or 1:1000 or lesser/greater, and/or any ranges between any combination of the above.

If desired, the material mixture additive may comprise one or more pharmaceutically and/or biologically active compounds. These may be incorporated into the micropores and/or mid-pores and in use may be used to stimulate cell growth around and into the biomaterial. For example, growth factors, such as transforming growth factor (TGF-βI), bone morphogenetic protein (BMP-2) or osteogenic protein (OP-I) maybe incorporated into the biomaterial. Further materials such as enzymes, vitamins (including Vitamin D) and trace material such as zinc (for example in the form of salt) may also be incorporated.

The ceramic material used to produce the granules may be any non-toxic ceramic known in the art, but in various embodiments will comprise a silicon nitride material and/or its chemical analogues.

In another exemplary embodiment, the bulk or carrier material of the implant may desirably be located primarily between the granules of silicon nitride, such that the carrier material adheres the adjacent granules together without completely encompassing each of the granules. In such a case, the resulting implant “block” or implant may have a spongy or swiss-cheese-like appearance.

In various embodiments, disclosed are biomaterials obtainable by various manufacturing processes. Bone implants, dental implants or ear, nose or throat (ENT) implants comprising both substitute materials according to the invention are also provided. Additionally, the invention provides the use of the bone substitute as a bone replacement, in dental implants or maxillofacial repair materials for the repair of bone breaks or fractures, osteoporotic bone, intervertebral space implants, and/or as a load bearing surface replacement on a bone.

In one exemplary embodiment, a silicon nitride material can be pulverized, for example using a ball mill or other milling machinery. The size of the resulting silicon nitride granules may be adjusted, for example, by sieving through a mesh of the desired size to regulate the size of the granules. The granules can then be mixed with an implant material which may similarly be powdered and/or granularized, in a similar manner to adding aggregate to cement to form concrete.

In the exemplary embodiment, a weight ratio of implant material to the silicon nitride material granules may be between 100:1 to 1:100, especially 10:1 to 1:10, and more preferably 1:1.

In various embodiments the silicon nitride granules and/or other material components thereof can be formed using a variety of techniques, including by compressing, milling and firing silicon nitride powder, as well as by extruding silicon nitride into sheet, tube, pipe and/or thread form (which may be further processed into thread or “rope” by braiding and/or other techniques). Silicon nitride shapes may also be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, silicon nitride may even be formed using curing or other light/energy activation techniques, such as where a slurry of liquid polymer and silicon nitride particles may be UV cured to create a 3-dimensional structure and/or layer containing silicon nitride. In various embodiments, silicone nitride may be utilized in block form, in sheets, columns and bars, in cable or braided form, in mesh form, in a textured surface coating, in powder form, in granular form, in gel, in putty, in foams and/or as a surface filler and/or coating. In some cases, a surface layer of silicon nitride may be formed, placed and/or deposited on an external and/or internal surface of an implant.

Once implanted in a desired location, an implant incorporating silicon nitride will desirably be highly osteo-inductive and/or osteoconductive and will desirably facilitate and/or promote fixation of the implant to adjacent living bone surfaces, while concurrently reducing and/or inhibiting periprosthetic infection and/or bacterial adhesion to the surfaces and/or interior portions of the implant.

If desired, a bone implant could be constructed from a variety of modular components, including at least one component comprising a silicon nitride material. If desired, such an implant and/or the components thereof could be provided in a kit form for selection and/or assembly in a surgical theatre and/or in situ during a surgical procedure. If desired, various components may be removable and replaceable.

In accordance with another aspect of the present subject matter, various methods for manufacturing an implant comprising a material mixture including silicon nitride and/or other materials, as set for within any of the details described with the present application, are provided.

While embodiments and applications of the present subject matter have been shown and described, it would be apparent that other embodiments, applications and aspects are possible and are thus contemplated and are within the scope of this application.

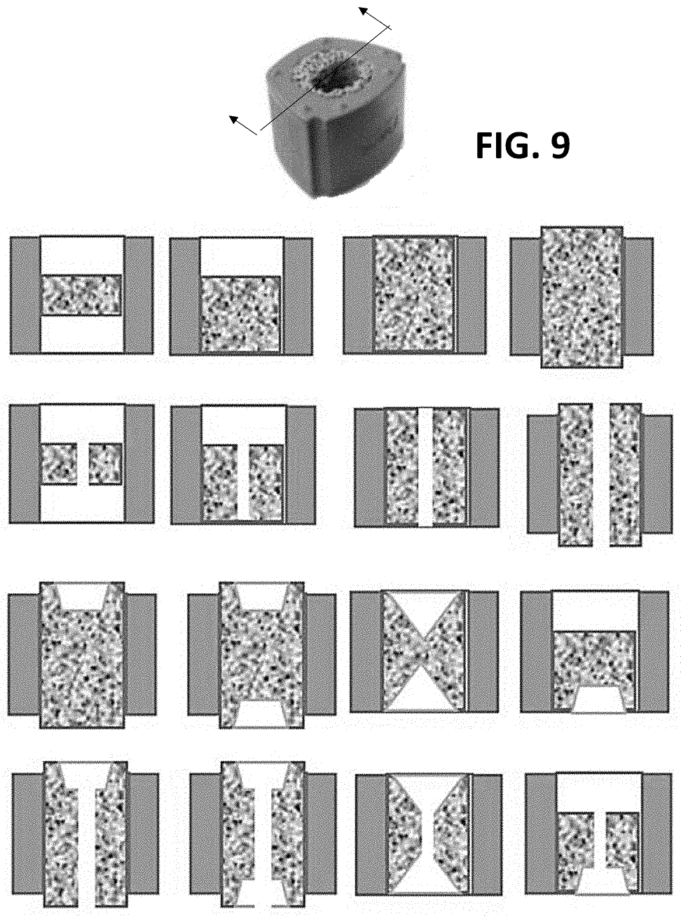

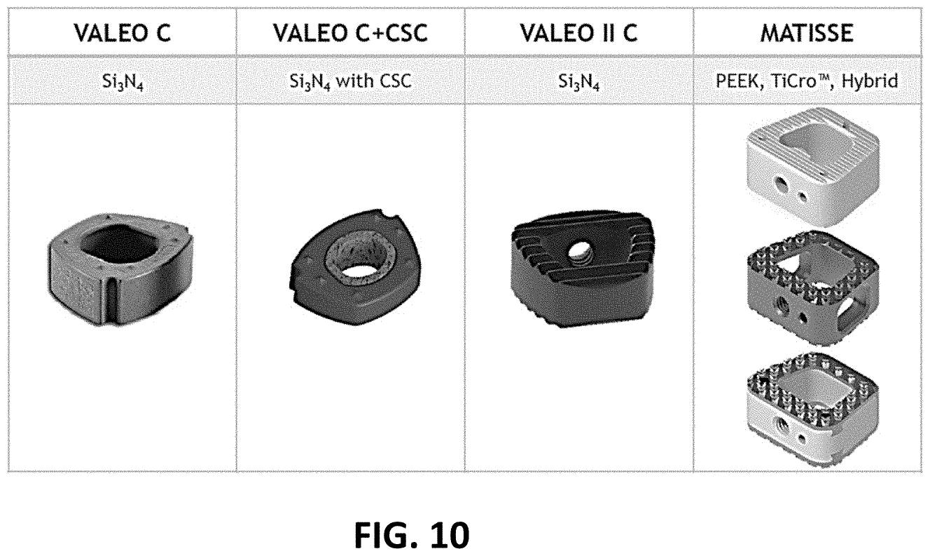

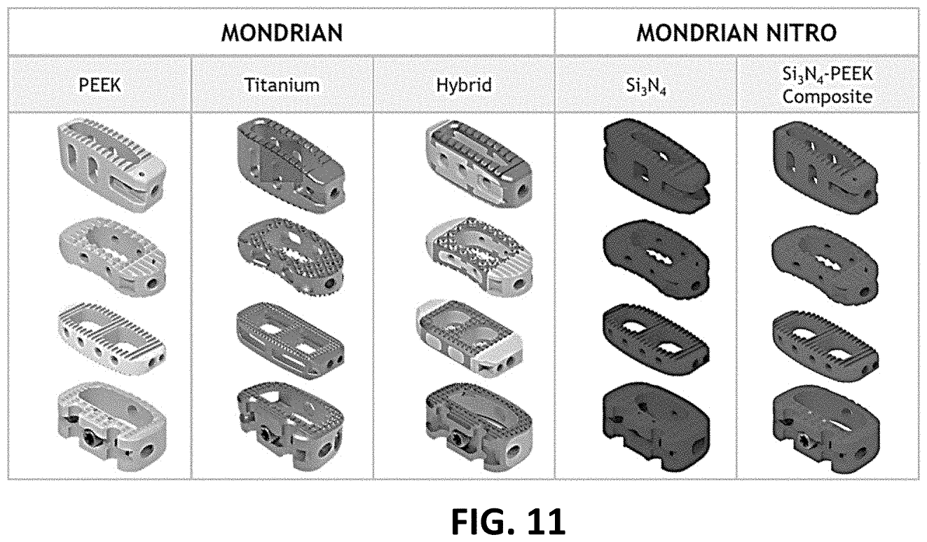

As previously noted, the various bone implants and/or components thereof disclosed herein can incorporate a silicon nitride material (i.e., Si₃N₄ and/or chemical analogues thereof) in their construction, either in the entirety of the implant as well as components, portions, layers, fillings, dispersed particulates and/or surfaces thereof. The incorporation of silicon nitride as a component material for spinal or other implants can provide significant improvements over existing implant materials and material designs currently available, as the silicon nitride material(s) will desirably be highly osteo-inductive and/or osteoconductive and will facilitate and/or promote implant fixation to adjacent living bone surfaces, while concurrently reducing and/or inhibiting periprosthetic infection and/or bacterial adhesion to the surfaces and/or interior portions of the implant. In various embodiments, materials including silicon nitride materials of differing compositions and/or states (i.e., solid, liquid and/or flowable or moldable “slurry” states, for example) could be utilized in a single implant and/or portions thereof, including the use of solid silicon nitride for an arthroplasty cage implant, with a moldable silicon nitride “paste” placed within a centrally positioned “graft chamber” of the implant. If desired, an implant could include some portion or insert formed from a silicon nitride material, wherein the silicon nitride or similar component could extend completely through an implant, or only extend partially into and/or out of an implant. For example, FIG. 9 depicts various cross-sectional views of spinal implants with various exemplary silicon nitride insert geometries formed therein, which can include the introduction of such silicon nitride materials in an uncured form which can then cure in situ, if desired.

In various embodiments the disclosed implants may incorporate materials such as silicon nitride that are “phase stable” to a desired degree. For example, various embodiments may desirably withstand standard autoclave sterilization conditions such as 120° C. 1 atmosphere steam for up to 100 hours of time, with no appreciable change in phase composition, no appreciable change in flexural strength and an inherently stable microstructure. Moreover, such materials could desirably provide favorable imaging characteristics, such as high levels of radiolucency and/or no significant MRI or CT scan artifacts.

In various embodiments, a mixed material implant may display varying degrees of hydrophobicity for various medical grade materials, including silicon nitride in various forms. Silicon nitride can be much less resistant to water penetration than other materials, which can be a highly desirably characteristic in many applications. If desired, an implant comprising silicon nitride can induce neovascularization within the porous sections of the implant, including internal pores colonized with mineralized bone to depths exceeding 5.5 mm or more.

FIGS. 12 and 13 depict some exemplary implants that can be constructed of ceramics, plastics, metals and/or any combinations thereof, in accordance with the present invention. In many instances, a pure PEEK implant may often be accompanied by surgical bone defects that do not fill in with new bone over time, as well as potential infection sites proximate to the implant that may be difficult or impossible to resolve (potentially necessitating implant removal in some cases). In a similar manner, bone infection sites near solely titanium implants can also be difficult or impossible to resolve, and may similarly necessitate implant removal. However, with a mixed material implant incorporating a silicon nitride material, the surface chemistry of the implant can actively destroy infectious bacterial agents, and also induce new bone growth immediately upon implantation. In essence, the effect of the silicon nitride material on new bone growth may act like a magnet on ferrous materials, actively “drawing” new bone near and into the implant.

Another significant advantage of using silicon nitride materials in bone implants is the anti-bacterial effects of the material on infectious agents. Upon implantation, a silicon nitride surface can induce an inflammatory response action which attacks bacterial biofilms near the implant. This reaction can also induce the elevation of bacterial pods above the implant surface by fibrin cables. Eventually the bacteria in the vicinity of the silicon nitride implant surfaces will be cleared by macrophage action, along with the formation of osteoblastic-like cells. In various experiments involving comparisons between standard implants and silicon nitride implants (both bulk and silicon nitride coated implants of standard materials), cell viability data in (which were determined at exposure times of 24 and 48 hours) showed the existence of a larger population of bacteria on the standard medical materials as compared to Si₃N₄ implants (both coated and bulk). A statistically validated decreasing trend for the bacterial population with time was detected on both coated and bulk substrates, with a highest decrease rate on Si₃N₄-coated substrates. Moreover, the fraction of dead bacteria at 48 hours was negligible on the standard implants, while almost the totality of bacteria underwent lysis on the Si₃N₄ substrates. In addition, optical density data provided a direct assessment of the high efficacy of the Si₃N₄ surfaces in reducing bacterial adhesion.

In various embodiments, the disclosed material mixtures can provide various combinations of significant advantages and desirable attributes of an abiotic spinal spacer or similar implant, such as one or more of the following: biocompatibility, mechanical integrity, radiological traceability, osteoconductivity, osteoinductivity and/or bacteriostasis. In various embodiments, silicon nitride materials can be incorporated into a variety of implants and implant-like materials, including (1) orthopedic bone fusion implants (i.e., screws, cages, cables, rods, plugs, pins), (2) dental implants, (3) cranial/maxillofacial implants, (4) extremity implants, (5) hip and joint implants, (6) bone cements, powders, putties, gels, foams, meshes, cables, braided elements, and (7) bone anchoring elements and/or features.

In various embodiments, a material mixture can be manufactured and/or molded into various shapes and/or sizes, which could include placement and/or incorporation into and/or around a shaft or other feature of a bone screw or other surgical implant. In some cases, because the material mixture may experience some level of shrinkage or otherwise deform during portions of the manufacturing and/or curing process, it may be desirable that the implant design features accommodate potential changes in the dimensions and/or density. In at least one exemplary embodiment, a silicon nitride material mixture may be mixed and molded in a sleeve or other shape, with a corresponding metal implant shape receiving the material mixture (either in a fully, partially and/or uncured condition).

In various embodiments, a surgical tool kit could include an implant comprising silicon nitride as one or more modular components for the system, including fully formed individual silicon nitride components, if desired. The various components of these systems could optionally be provided in kit form, with a medical practitioner having the option to select an appropriately sized and/or shaped implant and/or modular components to address a desired surgical situation.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

Of course, method(s) for manufacturing the various material mixture formulations and implants, including silicon nitride components and/or surgical devices and related components and implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

As previously noted, the use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An interbody system for implanting between vertebrae, comprising:
a cage having a cage body comprising at least one material from the group consisting of titanium, chrome cobalt, stainless steel, silicone, poly (ether ether ketone) (PEEK), ultra-high molecular-weight polyethylene (UHMWPE), polyurethane foam, polylactic acid and apatite, the cage body having at least one externally facing surface, and
a dispersed particulate layer of silicon nitride at least partially disposed within the cage body.

2. The interbody system of claim 1, wherein at least a portion of the dispersed particulate layer of silicon nitride is exposed on the at least one externally facing surface of the cage body.

3. The interbody system of claim 1, wherein at least a portion of the dispersed particulate layer of silicon nitride is exposed on an internally facing surface of the cage body.

4. The interbody system of claim 1, wherein at least a portion of the dispersed particulate layer of silicon nitride is encased within the cage body.

5. The interbody system of claim 1, wherein the dispersed particulate layer of silicon nitride comprises a powdered silicon nitride particulate that is mixed with the at least one material prior to forming the cage body by injection molding.

6. The interbody system of claim 1, wherein the dispersed particulate layer of silicon nitride comprises a liquified silicon nitride particulate that is mixed with the at least one material prior to forming the cage body by injection molding.

7. The interbody system of claim 1, wherein the dispersed particulate layer of silicon nitride comprises a plurality of silicon nitride particles that are mixed with the at least one material prior to forming the cage body by 3D additive manufacturing.

8. The interbody system of claim 1, wherein the dispersed particulate layer of silicon nitride comprises a powdered silicon nitride particulate that is mixed with the at least one material prior to forming the cage body by injection molding.

9. The interbody system of claim 1, wherein the dispersed particulate layer of silicon nitride comprises a plurality of silicon nitride particles that are mixed with the at least one material prior to forming the cage body by casting.

10. A method of manufacturing an interbody cage for implanting between vertebrae, comprising the steps of:
mixing a plurality of particles of a first material with at least one second material from the group consisting of titanium, chrome cobalt, stainless steel, silicone, poly (ether ether ketone) (PEEK), ultra-high molecular-weight polyethylene (UHMWPE), polyurethane foam, polylactic acid and apatite to form a feedstock material,
processing the feedstock material in an injecting molding apparatus which injects the processed feedstock into a mold having an internal cavity formed in the shape of the interbody cage.

11. The method of claim 10, wherein the first material is selected from the group consisting of silicon nitride, transforming growth factor (TGF-βI), bone morphogenetic protein (BMP-2), osteogenic protein (OP-I), enzymes, vitamins and zinc.

12. The method of claim 11, wherein the first material comprises a powdered particulate.

13. The method of claim 11, wherein the second material comprises a powdered particulate.

14. The method of claim 10, wherein the first material and second material are mixed within a liquified slurry.

15. The method of claim 11, further comprising the steps of:
removing an injection molded interbody cage from the internal cavity of the mold, and sintering the injection molded interbody cage.

16. The method of claim 11, further comprising the steps of:
removing an injection molded interbody cage from the internal cavity of the mold, machining at least a portion of the injection molded interbody cage; and
sintering the injection molded interbody cage.

17. The method of claim 11, further comprising the steps of:
removing an injection molded interbody cage from the internal cavity of the mold, adding at least one additional layer of material to at least a portion of the injection molded interbody cage by 3D additive manufacturing; and

18. An interbody system for implanting between vertebrae, comprising:
a cage having a cage body comprising at least one material selected from the group consisting of titanium, chrome cobalt, stainless steel, silicone, poly (ether ether ketone) (PEEK), ultra-high molecular-weight polyethylene (UHMWPE), polyurethane foam, polylactic acid and apatite, and
a dispersed particulate mixture of a second material from the group consisting of silicon nitride, transforming growth factor (TGF-βI), bone morphogenetic protein (BMP-2), osteogenic protein (OP-I), enzymes, vitamins and zinc.

19. The interbody system of claim 18, wherein the at least one material and the dispersed particulate mixture of the second material comprise a powdered particulate admixture prior to forming the cage body by an injection molding process.

20. The interbody system of claim 19, wherein the cage body undergoes a subsequent sintering process after the injection molding process.
